(19) United States
(12) Plant Patent Application Publication
(10) Pub. No.: US 2026/0130305 P1
(43) Pub. Date: May 7, 2026

(54) Hop plant Named 'HBC 1324'

(50) ;

(71) Applicant: Hop Breeding Company, L.L.C., Yakima, WA (US)

(72) Inventors: Eugene G. Probasco, Yakima, WA (US); Jason Perrault, Toppenlsh, WA (US); Scott Varnum, Selah, WA (US); Michael D. Ferguson, Yakima, WA (US); Jamie Avalos P., Granger, WA (US); Ryan W. Christian, Yakima, WA (US)

(21) Appl. No.: 18/831,796

(22) Filed: Sep. 10, 2025

Related U.S. Application Data
(60) Provisional application No. 63/732,795, filed on Sep. 18, 2024.

(51) Int. Cl. A01H 6/28; A01H 5/00

(52) U.S. Cl. CPC A01H 6/28; A01H 5/00
USPC PLT/236

(57) ABSTRACT

A new hop plant named ‘HBC 1324’ is disclosed. ‘HBC 1324’ is used for its exceptional and unique aromatic qualities, and is suitable for beer flavoring.

### CROSS-REFERENCE TO RELATED APPLICATIONS

Priority is claimed with respect to U.S. Provisional Patent Application 63/732,795 filed on Sep. 18, 2024.

### STATEMENT REGARDING FEDERALLY SPONSORED RESEARCH OR DEVELOPMENT

None

Genus and species:

Humulus lupulus.

Variety denomination:

‘HBC 1324’.

### BACKGROUND OF THE INVENTION

‘HBC 1324’ is a product of a controlled breeding program carried out by the co-inventors in the Yakima Valley of Washington State (WA). ‘HBC 1324’ was one of several seedlings resulting from a controlled cross made in 2012 with female parent ‘HBC 366’ that is a variety patented under U.S. Plant Pat. No. 25,899, and the unpatented male parent ‘50-61’. Seedling plants from this cross were planted in 2019 and screened for disease resistance, vigor, alpha acid content, and sex in field nursery near Granger, WA. Several individual selections were further evaluated in a greenhouse for disease resistance in 2020, and a single plant was selected, assigned an internal selection I.D. of 12.001.501 in 2020 and asexually reproduced via softwood cuttings, and expanded to 7 plants in an evaluation block near Toppenish, Wash. In 2021 the variety was selected for stage 2 yield trials and expanded to a triplicate multi-hill yield trial of 21 plants in an evaluation block near Toppenish, Wash. In 2022 the variety was expanded to 150 plants in a stage 3 trial near Toppenish, Wash. In 2023, the 12.001.501 plant was assigned the cultivar accession number ‘HBC 1324’ and expanded to 41 acres across multiple test blocks in Washington State, specifically near Toppenish, Granger, Sunnyside, and Wenas, with plants asexually reproduced via softwood cuttings. In 2024, the cultivar was expanded to 88 acres across multiple test blocks in Washington State, specifically near Toppenish, Granger, Sunnyside, and Wenas, with plants asexually reproduced via softwood cuttings. The ‘HBC 1324’ plants have now been observed and evaluated for several years. Throughout several generations of asexual propagation, ‘HBC 1324’ has been observed to retain its distinctive characteristics and remain true to type.

### SUMMARY OF THE INVENTION

A new and distinct hop plant is described herein, with the ‘HBC 1324’ cultivar characterized by having cones with a high alpha acid content. The new cultivar exhibits onion and garlic, grassy, and herbaceous aromas. The new cultivar is suitable for beer flavoring.

### BRIEF DESCRIPTION OF THE PHOTOGRAPHS

FIG. 1 illustrates mature bines of a ‘HBC 1324’ hop plant on a trellis at approximately four months of vegetative growth;

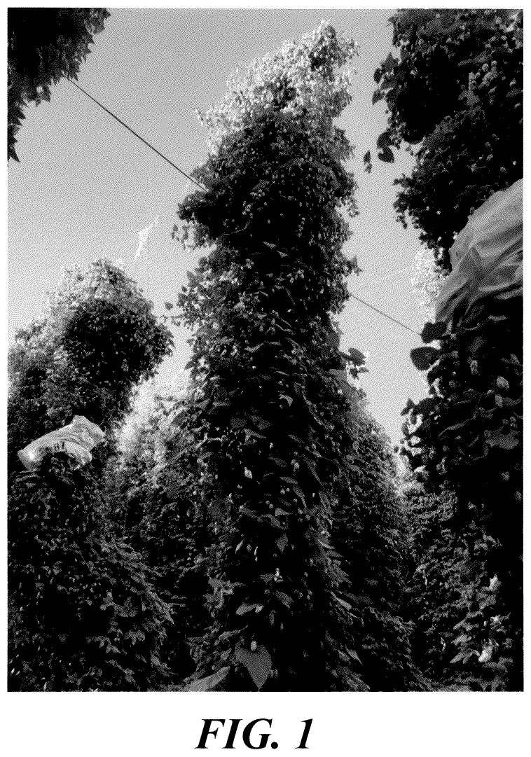

FIG. 2 illustrates mature leaves and cones of a ‘HBC 1324’ hop plant at approximately four months of vegetative growth;

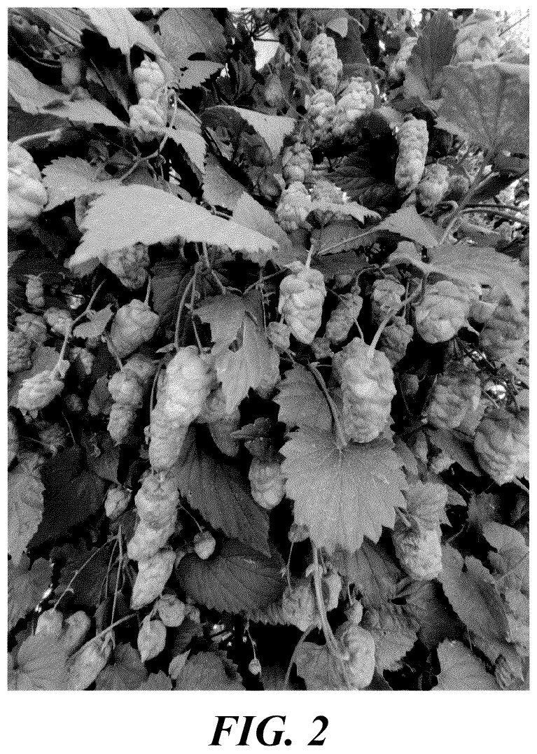

FIG. 3 illustrates a close up of a mature bine of a ‘HBC 1324’ hop plant at approximately four months of vegetative growth;

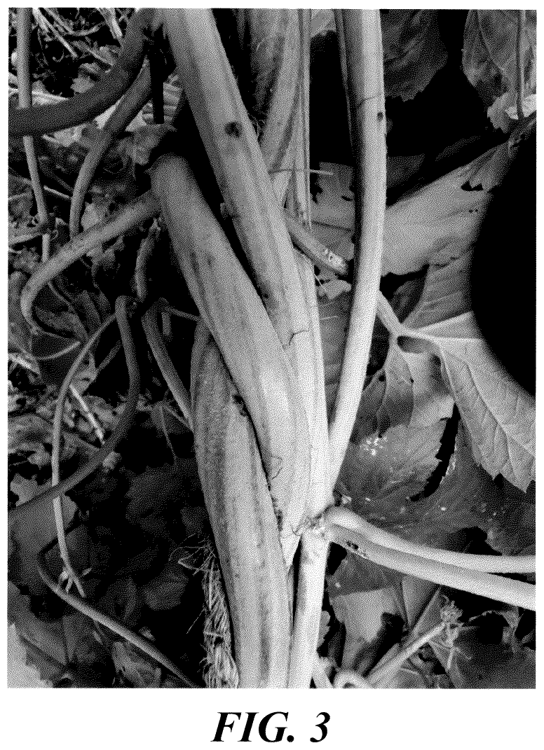

FIG. 4 illustrates a close up of cross-sectioned mature cones of a ‘HBC 1324’ hop plant at approximately four months of vegetative growth; and

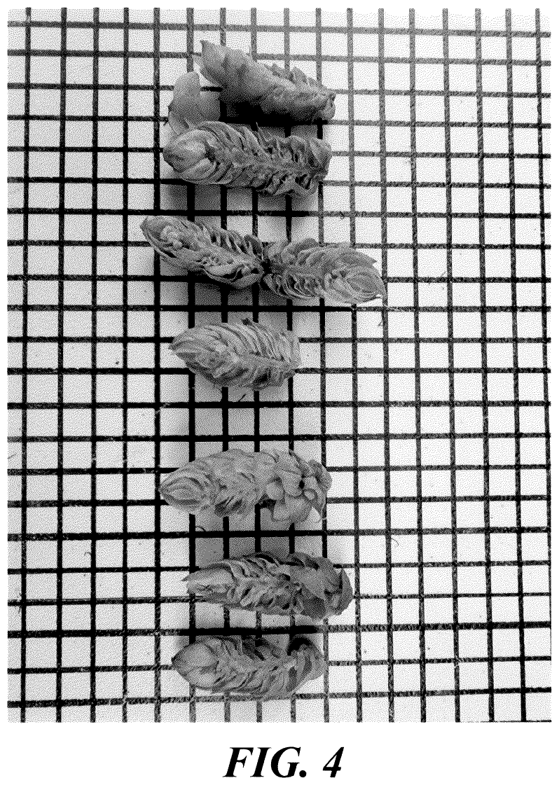

FIG. 5 illustrates a close up of a mature leaf of a ‘HBC 1324’ hop plant at approximately four months of vegetative growth.

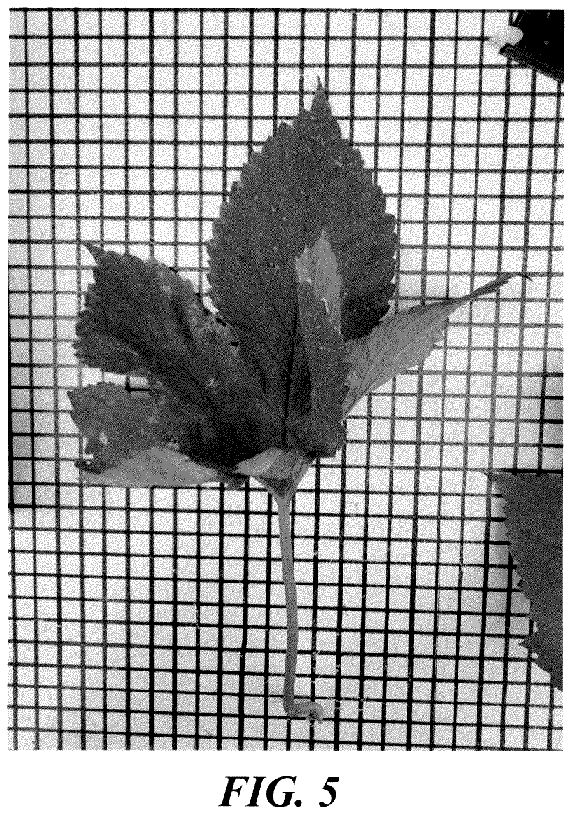

The colors of these illustrations may vary with lighting conditions and, therefore, color characteristics of this new variety should be determined with reference to the observations described herein, rather than from these illustrations alone.

### COMPARISON OF ‘HBC 1324’ TO PARENT PLANT AND COMARISON CULTIVAR

‘HBC 1324’ though having a distinctive onion and garlic aroma, it has other aroma and flavor characteristics similar to its female parent, the patented cultivar ‘HBC 366’ (U.S. Plant Pat. No. 25,899). The cones of ‘HBC 1324’ are distinguished from ‘HBC 366’ by typically having a higher alpha acids content. Additionally, ‘HBC 1324’ is more vigorous and higher yielding, as compared to ‘HBC 366’.

Table 1. below, sets forth some of the distinguishing characteristics of ‘HBC 1324’ as typically observed, as compared to its female parent ‘HBC 366’, and to the ‘COLUMBUS’ cultivar as a closely comparable cultivar.

TABLE 1

| | | FEMALE | COMPARISON |
|---|---|---|---|
| | INSTANT CV. | PARENT | CV. |
| | ‘HBC 1324’ | ‘HBC 366’ | ‘COLUMBUS’ |
| Alpha | 12.0-19.8 | 14.4-17.9 | 14.5-17.5 |
| (% of cone weight) | | | |
| Beta | 3.4-6.3 | 4.0-5.5 | 4.2-5.9 |
| (% of cone weight) | | | |
| Co-humulone | 27.0-27.2 | 31.7-38.0 | 24.9- 30.2 |
| (% of alpha acids) | | | |
| Total Oil (mL/100 g) | 2.5-2.7 | 2.5 -4.5 | 2.0-4.0 |
| Aroma Profile | onion and garlic, | citrus, stone | earthy, citrus, |
| | grassy, and | and tropical | earthy, woody |
| | herbaceous | fruit, woody | |

### DETAILED BOTANICAL DESCRIPTION

The following description is based on observations made in September of 2023 near Granger, Washington, on mature plants at approximately five months of vegetative growth. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant or any group of plants, of the new variety may vary from the stated average. Color code designations are by reference to the R.H.S. Colour Chart, 4th ed., Copyright 2001, published by The Royal Horticultural Society of Great Britain.
Ploidy: Diploid.
Sex: Female.
Use: Brewing beer and other hop flavored beverages.
Yield: Average yield is expected to be approx. 3,600 pounds per acre.
Plant shape: Clavate.
Growth type: Normal.
Disease and pest resistance or susceptibility:
   Powdery Mildew (Podosphaera macularis).—Resistant.
   Downy Mildew (Plasmopara halstedii).—Tollerant.
Hardiness: Observed Plant Hardiness Zone 7a to 6b .
Freeze tolerance: Typically intolerant of freezing temperatures during the growing season.
Table of oil analytical characteristics (observed averages):

TABLE 2

| | |
|---|---|
| % Alpha Acid (% cone weight) | 17.1 |
| % Beta Acid (% cone weight) | 5.4 |
| % Cohumulone (% alpha acids) | 27.1 |
| % Myrcene (% of total oils) | 34.0 |
| % Caryophyllene (% of total oils) | 6.1 |
| % Farnesene (% of total oils) | 0.0 |
| % Linalool (% of total oils) | 0.3 |
| Total Oil (ml/100 g of cones): | 2.6 |
| Hop Storage Index (Approx. % loss of | 34 |
| Alpha Acids at 6 months) | |

Bine:
   Bine color.—Yellow-Green 149A.
   Stripe present.—Yes.
   Stripe color.—Greyed-Purple 184B.
   Stipule direction.—Up and down.
   Stipule color.—Yellow-Green 144B.
   Average stipules per bine.—263.
   Typical bine length.—7 m.
   Typical internode length on bine.—18 cm to 20 cm.
   Typical internode length on lateral.—5 cm to 15 cm.
   Bine diameter.—10.5 mm at base; 10.7 mm at nine feet; and 9.3 mm at terminal end or eighteen feet.
   Average lateral length (on middle third of plant).—1.2 m.
   Average laterals length (on upper third of plant).—1.3 m.
   Lateral density (on middle third of plant).—Medium (two laterals per node).
   Lateral density at middle third of plant.—Two laterals per Node.
   Anthocyanin coloration on main bine.—Weak.
Leaf:
   Arrangement.—Opposite.
   Shape.—Palmately Lobed.
   Apex.—Aristulate.
   Base.—Cordate.
   Margin.—Serrate.
   Serrations per inch (typical).—5.
   Pose.—Down.
   Average length of mature leaf.—12.3 cm.
   Average width of mature leaf.—19.4 cm.
   Leaf blistering on the upper side of leaf blade.—Present.
   Color of mature leaf upper surface.—Yellow-Green 147A.
   Color of mature leaf lower surface.—Yellow-Green 147A.
   Color of immature leaf upper surface.—Yellow-Green 146A.
   Color of immature leaf lower surface.—Yellow-Green 146A.
   Typical Number of lobes.—3 to 5.
   Petiole color at base.—Yellow-Green 147D.
   Typical petiole diameter (mature).—3.7 mm.
   Typical petiole length (mature).—7.0 cm.
   Venation.—Palmate.
   Vein color.—Yellow-Green 147D.
Cone:
   Cone shape.—Oblong to Rotund.
   Average cone length.—3.5 cm.
   Average cone diameter.—1.7 cm.
   Average cone weight.—0.94 g.
   Bract shape.—Cuspidate.
   Bract base shape.—Rounded.
   Bract margin.—Entire.
   Bract tip shape.—Cuspidate.
   Bract tip position.—Recurved Downward.
   Degree of bract opening.—Closed.
   Bract tip color.—Yellow-Green 144A.
   Bract base color.—Yellow-Green 144D.
   Typical bract length.—14 mm.
   Typical bract width.—15 mm.
   Bracteole shape.—Ovate.
   Typical bracteole length.—12.2 mm.
   Typical bracteole width.—8.5 mm.
   Bracteole color (upper surface).—Yellow-Green 149B.
   Bracteole color (lower surface).—Yellow-Green 149B.
   Lupulin glands shape.—Pedunculated Oblong Polyps.
   Lupulin glands color.—Yellow-Green 7A.
   Number of lupulin glands per cone.—Varies Greatly (Moderate to Many), with approximately 50,000 lupulin glands per Cone.
   Pickability.—Good.
   Time of flowering.—Late July.
   Maturity date range.—Medium to Late.
   Harvest dates.—Typically harvested September 21 to September 26, but can deviate from this time period, depending on growing practices and location.

What is claimed:

1. A new and distinct variety of Hop plant named ‘HBC 1324’, substantially as illustrated and described herein.
